(19) United States
(12) Patent Application Publication
(10) Pub. No.: US 2024/0238465 A1
(43) Pub. Date: Jul. 18, 2024

(54) REPROCESSING SYSTEM FOR A SURGICAL INSTRUMENT AND METHOD FOR OPERATING A REPROCESSING SYSTEM

(71) Applicant: OLYMPUS Winter & Ibe GmbH, Hamburg (DE)

(72) Inventor: Stefan SCHROEDER, Hamburg (DE)

(73) Assignee: OLYMPUS Winter & Ibe GmbH, Hamburg (DE)

(21) Appl. No.: 18/390,726

(22) Filed: Dec. 20, 2023

Related U.S. Application Data
(60) Provisional application No. 63/439,626, filed on Jan. 18, 2023.

(51) Int. Cl. A61L 2/24

(52) U.S. Cl. CPC A61L 2/24; A61L 2202/122; A61L 2202/14; A61L 2202/24

(57) ABSTRACT

A reprocessing system including: a reprocessing device for a surgical instrument, wherein the reprocessing device has a cabinet for accommodating the surgical instrument within the cabinet; a sensor configured to detect at least one or more non-contact operating gesture patterns of a reprocessing device operator for the reprocessing device; and a controller comprising hardware, the controller being configured to: receive an output from the sensor indicating detection of the at least one or more non-contact operating gesture patterns of the reprocessing device operator for the reprocessing device; and operate the reprocessing device based on the output from the sensor.

### CROSS-REFERENCE TO RELATED APPLICATION

The present application is based upon and claims the benefit of priority from U.S. Provisional Application No. 63/439,626 filed on Jan. 18, 2023, the entire contents of which is incorporated herein by reference.

### BACKGROUND

### Field

The present disclosure relates to a reprocessing system, in particular a reprocessing system for use with a surgical instrument. Furthermore, the present disclosure relates to a method for operating a reprocessing system.

### Prior Art

Reprocessing devices for surgical instruments, for example the ETD series of Olympus, generally have a washing cabinet for receiving surgical instruments such as endoscopes to be reprocessed. In the washing cabinets, there are for example baskets for receiving the endoscopes. Adapters are provided in the washing cabinet with which the endoscope channels can be connected to supply channels of the endoscope reprocessing device. During reprocessing, the reprocessing device circulates a reprocessing fluid through the various endoscope channels. The reprocessing fluid is for cleaning and disinfecting the inner endoscope channels after use. After completion of the cleaning and disinfection routine, reprocessing of the endoscope continuous with switching over to the drying routine. During drying, the endoscope channels are flushed with air. The drying air is often supplied by a pressurized air source.

The operation of such reprocessing devices require an interaction between an operating personal and the device, especially for opening or closing the door of the reprocessing device. For loading or unloading the reprocessing devices, an operator handles the endoscope with his/her hands and operates the reprocessing device manually in order to start the reprocessing procedure for the surgical instruments. Due to the manual physical interaction of the operator with the reprocessing device, surfaces of the reprocessing device will be contaminated with germs, like bacteria etc.

### SUMMARY

An object is to reduce the transmission of germs in the environment of reprocessing device for surgical instruments.

Such object can be solved by a reprocessing system, such as a reprocessing device for surgical instruments, comprising:
a reprocessing device for a surgical instrument, such as an endoscope, wherein the reprocessing device has a cabinet for accommodating the surgical instrument within the cabinet, and
a controller comprising hardware, the controller being configured to control the reprocessing device; and
a gesture pattern detection radar sensor, wherein the gesture pattern detection radar sensor is connected with the controller,
wherein the gesture pattern detection radar sensor is configured to detect at least one or more non-contact operating gesture patterns of a reprocessing device operator for the reprocessing device, and
the controller is configured to operate the reprocessing device upon the at least one recognition of the at least one or more non-contact gestures of the reprocessing device operator, detected by the gesture pattern detection radar sensor.

Therefore, operating gestures of a reprocessing device operator can be detected by a gesture pattern detection radar sensor in order to operate the reprocessing device without contact. The gesture pattern detection radar sensor can be implemented in a room for reprocessing the surgical instruments. Moreover, in another embodiment, the reprocessing device can be provided with the gesture pattern detection radar sensor. With such gesture pattern detection radar sensor, different conditions in a certain area in front of the reprocessing device can be detected.

For example, it is possible to detect various situations in front of the reprocessing device, such as:
a device operator without a basket or an endoscope,
a device operator without a basket but with an endoscope,
a device operator with a basket but without an endoscope, and/or
a device operator with a basket and with an endoscope.

Moreover, the gesture pattern detection radar sensor can be configured to also detect various situations for operating the reprocessing device by contactless commands executed by a device operator, such as:
a device operator indicating a number by putting up one finger,
a device operator indicating a number by putting up two fingers, and/or
a device operator indicating a number by putting up three fingers.

The various situations can be identified or recognized by the gesture pattern detection radar sensor, wherein an individual action or operation of the reprocessing device can be possible based on such identification or recognition.

A benefit of the reprocessing system for surgical instruments can be that a proper functioning of the reprocessing system can be maintained in a complex environment containing different temperatures, humidity, noise, air current, dust, light etc. Irrespective of the conditions of the environment, the reprocessing system can be operated without contact by the gestures of a reprocessing device operator, wherein the contamination of germs on the surfaces of the reprocessing system or the reprocessing device can be significantly reduced.

According to another embodiment, the gesture pattern detection radar sensor can comprise an artificial intelligence module, wherein the artificial intelligence module can be configured to recognize at least one or several operating gesture patterns of the reprocessing device operator.

In another embodiment, the artificial intelligence module can be configured to learn and/or to identify several operating gesture patterns of at least two or more reprocessing device operators.

The artificial intelligence module can comprise a machine learning algorithm for analyzing and/or recognizing the at least one or more non-contact gestures of the reprocessing device operator or reprocessing device operators.

Using a machine learning algorithm, the operation of the gesture pattern detection radar sensor can be adapted to different reprocessing device operators and/or different conditions in the environment of the reprocessing system.

The artificial intelligence module can comprise a gesture pattern detection algorithm for recognizing several individual gesture patterns of at least one reprocessing device operator.

In one embodiment, the gesture pattern detection radar sensor can be configured to detect at least of one or more gestures of the reprocessing device operator without the surgical instrument and/or at least one or more gestures of the reprocessing device operator with the surgical instrument.

Furthermore, the gesture pattern detection radar sensor can be configured to detect at least one or more gestures of the reprocessing device operator with a nonsurgical device and/or at least one or more gestures of the reprocessing device operator without a nonsurgical device.

The nonsurgical device can be provided as a container for a surgical instrument, a basket for a surgical instrument, or as an adapter for a surgical instrument.

In an embodiment, the gesture pattern detection radar sensor can be provided as a double radar sensor unit.

The reprocessing device can be provided as a storage container for storage and/or transport of a surgical instrument, which can be disinfected, or the reprocessing device can be provided as a cleaning and disinfection device for reprocessing a surgical instrument.

The controller can be configured to open a door of the reprocessing device upon the at least one recognition of the at least one non-contact gesture of the reprocessing device operator, detected by the gesture pattern detection radar sensor and/or the controller can be configured to close the door of the reprocessing device upon the at least one recognition of the at least one non-contact gesture of the reprocessing device operator, detected by the gesture pattern detection radar sensor.

In one embodiment the controller can be configured to start and/or to perform a reprocessing procedure for reprocessing the surgical instrument upon the at least one recognition of the at least one non-contact gesture of the reprocessing device operator or the controller can be configured to stop the reprocessing procedure for reprocessing the surgical instrument upon the at least one recognition of the at least one noncontact gesture of the reprocessing device operator.

Such object can also be solved by a method for operating a reprocessing system for a surgical instrument, such as described above, wherein the reprocessing system can comprise:
a reprocessing device for a surgical instrument, such as an endoscope, wherein the reprocessing device has a cabinet for accommodating the surgical instrument within the cabinet, and
a controller comprising hardware, the controller being configured to control the reprocessing device; and
a gesture pattern detection radar sensor, wherein the gesture pattern detection radar sensor is connected with the controller,
wherein the gesture pattern detection radar sensor is configured to detect at least one or more non-contact operating gesture patterns of a reprocessing device operator for the reprocessing device, and
wherein the controller is configured to operate the reprocessing device upon the at least one recognition of the at least one or more non-contact gestures of the reprocessing device operator, detected by the gesture pattern detection radar sensor.

In one aspect, the gesture pattern detection radar sensor can comprise an artificial intelligence module,
wherein the artificial intelligence module can be configured to recognize at least one or several operating gesture patterns of the reprocessing device operator.

In another embodiment, the artificial intelligence module can be configured to learn and/or to identify several operating gesture patterns of at least two or more reprocessing device operators.

The artificial intelligence module can comprise a machine learning algorithm for analyzing and/or recognizing the at least one or more non-contact gestures of the reprocessing device operator or reprocessing device operators.

Furthermore, the artificial intelligence module can comprise a gesture pattern detection algorithm for recognizing several individual gesture patterns of at least one reprocessing device operator.

The gesture pattern detection radar sensor can detect at least of one or more gestures of the reprocessing device operator without the surgical instrument and/or at least one or more gestures of the reprocessing device operator with the surgical instrument.

The gesture pattern detection radar sensor can detect at least one or more gestures of the reprocessing device operator with a nonsurgical device and/or at least one or more gestures of the reprocessing device operator without a nonsurgical device.

In another aspect of the method, the nonsurgical device can be provided as a container for a surgical instrument, a basket for a surgical instrument, or as an adapter for a surgical instrument.

The gesture pattern detection radar sensor can be provided as a double radar sensor unit.

Moreover, the reprocessing device can be provided as a storage container for storage and/or transport of a surgical instrument, which can be disinfected, or the reprocessing device can be provided as a cleaning and disinfection device for reprocessing a surgical instrument.

The controller can open a door of the reprocessing device upon the at least one recognition of the at least one non-contact gesture of the reprocessing device operator, detected by the gesture pattern detection radar sensor and/or the controller can close the door of the reprocessing device upon the at least one recognition of the at least one non-contact gesture of the reprocessing device operator, detected by the gesture pattern detection radar sensor.

In another embodiment, the controller can start and/or perform a reprocessing procedure for reprocessing the surgical instrument upon the at least one recognition of the at least one non-contact gesture of the reprocessing device operator or the controller can stop the reprocessing procedure for reprocessing the surgical instrument upon the at least one recognition of the at least one noncontact gesture of the reprocessing device operator.

### BRIEF DESCRIPTION OF THE DRAWINGS

Further characteristics will become apparent from the description of the embodiments together with the claims and the included drawings. Embodiments can fulfill individual characteristics or a combination of several characteristics.

The embodiments are described below, without restricting the general intent of the invention, based on the exemplary embodiments, wherein reference is made expressly to the drawings with regard to the disclosure of all details that are not explained in greater detail in the text. In the drawings:

FIG. 1 illustrates a schematic simplified representation of a system for handling of a surgical instrument in a healthcare facility during a reprocessing cycle,

FIG. 2 illustrates schematically an illustration of a storage cabinet for endoscopes according to an embodiment.

In the drawings, the same or similar types of elements or respectively corresponding parts are provided with the same reference numbers in order to prevent the item from needing to be reintroduced.

### DETAILED DESCRIPTION

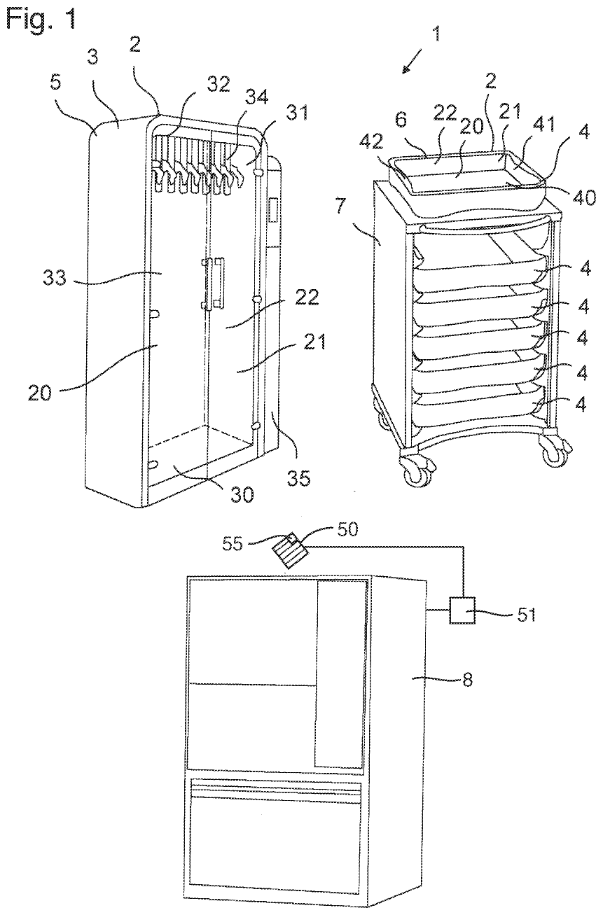

FIG. 1 shows a schematic representation of an exemplary embodiment of a system 1 for handling of a surgical instrument 10 in a healthcare facility during a reprocessing cycle. The surgical instrument 10 is not shown in FIG. 1. The system 1 comprises a plurality of care cycle apparatuses 5, 6 and a reprocessing apparatus 8. The first care cycle apparatus 5 is a storage cabinet 3 and the second care cycle apparatus 6 is a transport container 4. Both the storage cabinet 3 and the transport container 4 are containers 2 for accommodating surgical instruments 10.

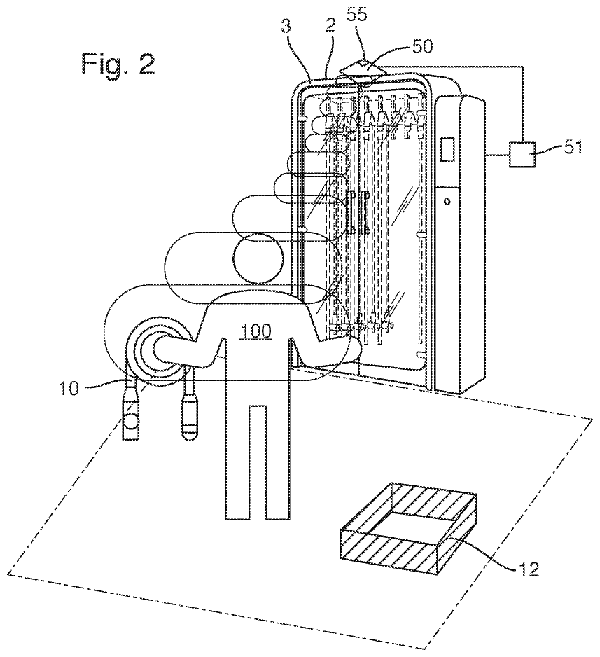

After use in surgery, the surgical instrument 10 (see FIG. 2) is cleaned and/or disinfected in the reprocessing apparatus 8. Afterwards it is transported with the transport container 4, which in this example is a tray, to the storage cabinet 3 for drying and storage. During transport, the surgical instrument 10 is placed in a storage space 20 of the transport container 4. The transport container 4 has a bottom wall 40, four side walls 41 and a top wall 42 at its upper edge. In the exemplary embodiment, the bottom wall 40 and the side walls 41 are the walls 21 confining the storage space 20. During transport, the surgical instrument 10 may come into contact with an inner surface 22 of these walls 21.

After receiving the surgical instrument 10, the transport container 4 is placed in a transport trolley 7, which can hold several transport containers 4. The transport trolley 7 has for example four wheels, so that the medical personal can move the stored transport containers 4 to the areas where they are needed.

For drying and storage of the surgical instrument 10, it is placed in the storage space 20 of the storage cabinet 3. The storage space 20 of the storage cabinet 3 comprises a bottom wall 30, three side walls 31, a top wall 32 and doors 33, which form the walls 21 of the storage space 20. Inside the storage space 20, a number of holders 34 are provided, which can hold the surgical instruments 10, for example flexible endoscopes. A drying attachment 35 helps with the drying of the surgical instruments 10 in the storage space 20.

FIG. 2 shows schematically an illustration of a storage cabinet 3 which is provided with a gesture pattern detection radar sensor 50 in order to detect noncontact operating gesture patterns of a reprocessing device operator 100 in front of the storage cabinet 3. Although the present embodiment is disclosed with regard to a radar sensor, those skilled in the art could implement such embodiment with an image sensor where performance of the image sensor is acceptable in the provided environment. The gesture pattern detection radar sensor 50 is connected with a controller 51 of the storage cabinet 3. Controller 51 can be configured as dedicated hardware circuits or a hardware processor executing a series of software instructions. The controller 51 is configured to control the storage cabinet 3. Using the control device 51, the door 33 of the storage cabinet 3 is opened or closed upon a corresponding operating gesture pattern of the reprocessing device operator 100. The controller 51 is configured to receive the recognized different operating gesture patterns of the reprocessing device operator 100, such as from a machine learning module integrated into the radar sensor 50, and to identify the operating gesture patterns. After identification of an operating gesture pattern by the controller 51, a specific command or function of the storage cabinet 3 is executed.

The radar senor 50 is configured to identify objects and contexts based on previously developed machine learning algorithms. For example, the radar sensor 50 and machine learning algorithm can identify reprocessing personal with reprocessing objects, such as endoscope types and/or accessories or with carried objects, such as baskets and/or trays. The radar senor 50 and machine learning algorithm can also identify reprocessing personal without the reprocessing objects or without the carried objects. With regard to contexts, the radar senor 50 and machine learning algorithm can identify a context or a plurality of contexts. Objects might be part of a context (e.g. reprocessing personal holding a basket in its hands), or not (e.g. a basket laying around or a person walking in front of the machine).

For example, different situations in front of the storage cabinet can be identified by integration of the gesture pattern detection radar sensor 51 like the reprocessing device operator 100 with an endoscope 10 and without a basket 12 (for an endoscope 10), as shown in FIG. 2 or the reprocessing device operator without the basket 12 and without an endoscope 10 etc. Furthermore, the gesture pattern detection radar sensor 50 is configured to detect also various situations for operating the reprocessing device by contactless commands executed by the reprocessing device operator 100 like indicating a number by putting up one finger for opening the door 33 of the storage cabinet 3 or indicating a number by putting up two fingers for closing the door 33 of the storage cabinet 3. In this regard, controller 51 sends an appropriate command to an actuator, such as a motor, which can be integrated into hinges attaching the door to the body of the storage cabinet 3, instructing the door to open or close.

The gesture pattern detection radar sensor 50 has an artificial intelligence module 55 in order to recognize at least one or several operating gesture patterns of the reprocessing device operator 100 in front of the storage cabinet 3. In a further embodiment, the controller 51 is provided with the artificial intelligence module 55. In an embodiment, the artificial intelligence module comprises a machine learning algorithm for analyzing and/or recognizing the contactless gestures of the reprocessing device operator 100 in front of the storage cabinet 3.

As shown in FIG. 1, the reprocessing apparatus 8 can also be provided with a gesture pattern detection radar sensor 50 comprising an artificial intelligence module 55. After recognition of contactless operating gesture patterns of a reprocessing device operator by means of the gesture pattern detection radar sensor 50, the reprocessing apparatus 8 can be opened or closed similarly with regard to the storage cabinet described above. In another embodiment, the reprocessing apparatus 8 can be started or stopped, via the controller 51, after detection of an operating gesture pattern of a reprocessing device operator in front of the reprocessing apparatus 8.

Using the gesture pattern detection radar sensor 50, the transmission of germs or cross-contaminations are reduced significantly for the operation of the storage cabinet 3 (see FIG. 2) or the reprocessing apparatus 8 (see FIG. 1). Moreover, the gesture pattern detection radar sensor 50 has a high robustness and maintains a proper functioning of the devices in complex environments containing different temperatures, humidities, noise, air currents, dust, light etc.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCES

1 system
2 container
3 storage cabinet
4 transport container
5 first care cycle apparatus
6 second care cycle apparatus
7 transport trolley
8 reprocessing apparatus
10 surgical instrument
12 basket
20 storage space
21 wall
22 inner surface
23 antimicrobial surface
30 bottom wall
31 side wall
32 top wall
33 door
34 holder
35 drying attachment
40 bottom wall
41 side wall
42 top wall
50 gesture pattern detection radar sensor
51 control device
55 artificial intelligence module
100 reprocessing device operator

What is claimed is:

1. A reprocessing system comprising:
a reprocessing device for a surgical instrument, wherein the reprocessing device has a cabinet for accommodating the surgical instrument within the cabinet;
a sensor configured to detect at least one or more non-contact operating gesture patterns of a reprocessing device operator for the reprocessing device; and
a controller comprising hardware, the controller being configured to: receive an output from the sensor indicating detection of the at least one or more non-contact operating gesture patterns of the reprocessing device operator for the reprocessing device; and
operate the reprocessing device based on the output from the sensor.

2. The reprocessing system according to claim 1, wherein:
the sensor comprises a gesture pattern detection radar sensor comprising an artificial intelligence module; and
the artificial intelligence module is configured to recognize at least one or several operating gesture patterns of the reprocessing device operator.

3. The reprocessing system according to claim 2, wherein the artificial intelligence module is configured to one or more of learn and identify several operating gesture patterns of at least two or more reprocessing device operators.

4. The reprocessing system according to claim 2, wherein the artificial intelligence module comprises a machine learning algorithm for one or more of analyzing and recognizing the at least one or more non-contact gestures of the reprocessing device operator.

5. The reprocessing system according to claim 2, wherein the artificial intelligence module comprises a gesture pattern detection algorithm for recognizing several individual gesture patterns of at least one reprocessing device operator.

6. The reprocessing system according to claim 1, wherein the sensor is configured to detect one or more of:
at least of one or more gestures of the reprocessing device operator without the surgical instrument; and
at least one or more gestures of the reprocessing device operator with the surgical instrument.

7. The reprocessing system according to claim 1, wherein the sensor is configured to detect one or more of:
at least one or more gestures of the reprocessing device operator with a nonsurgical device; and
at least one or more gestures of the reprocessing device operator without a nonsurgical device.

8. The reprocessing system according to claim 7, wherein the nonsurgical device is one of a container for a surgical instrument, a basket for a surgical instrument, or as an adapter for a surgical instrument.

9. The reprocessing system according to claim 1, wherein the sensor is a gesture pattern detection radar sensor.

10. The reprocessing system according to claim 1, wherein the reprocessing device is a storage container for one or more of storage and transport of the surgical instrument or the reprocessing device is a cleaning and disinfection device for reprocessing a surgical instrument.

11. The reprocessing system according to claim 1, wherein the controller is configured to one or more of:
open a door of the reprocessing device upon the at least one recognition of the at least one non-contact gesture of the reprocessing device operator, detected by the sensor and
close the door of the reprocessing device upon the at least one recognition of the at least one non-contact gesture of the reprocessing device operator detected by the sensor.

12. The reprocessing system according to claim 1, wherein the controller is configured to one of:
perform a reprocessing procedure for reprocessing the surgical instrument upon the at least one recognition of the at least one non-contact gesture of the reprocessing device operator; or
stop the reprocessing procedure for reprocessing the surgical instrument upon the at least one recognition of the at least one noncontact gesture of the reprocessing device operator.

13. A method for operating a reprocessing device for a surgical instrument, the method comprising:
recognizing at least one or more non-contact operating gesture patterns of a reprocessing device operator for the reprocessing device using a sensor; and
based on an output of the sensor to a controller, operating the reprocessing device with the controller based on upon the recognition.

14. The method according to claim 13, wherein:
the recognizing comprises using an artificial intelligence module, and
the artificial intelligence module is configured to recognize at least one or several operating gesture patterns of the reprocessing device operator.

15. The method according to claim 14, wherein the artificial intelligence module is configured to one or more of learn and identify several operating gesture patterns of at least two or more reprocessing device operator.

16. The method according to claim 14, wherein the artificial intelligence module comprises a machine learning algorithm for one or more of analyzing and recognizing the at least one or more non-contact gestures of the reprocessing device operator or reprocessing device operator.

17. The method according to claim 14, wherein the artificial intelligence module comprises a gesture pattern detection algorithm for recognizing several individual gesture patterns of the reprocessing device operator.

18. The method according to claim 13, wherein the recognizing detects one or more of:
at least of one or more gestures of the reprocessing device operator without the surgical instrument; and
at least one or more gestures of the reprocessing device operator with the surgical instrument.

19. The method according to claim 13, wherein the recognizing detects one or more of:
at least one or more gestures of the reprocessing device operator with a nonsurgical device; and
at least one or more gestures of the reprocessing device operator without a nonsurgical device.

20. The method according to claim 19, wherein the nonsurgical device is one of a container for a surgical instrument, a basket for a surgical instrument, or as an adapter for a surgical instrument.

21. The method according to claim 13, wherein the sensor comprises a gesture pattern detection radar sensor.

22. The method according to claim 13, wherein the reprocessing device is one of:
a storage container for one or more of storage and transport of a surgical instrument; or
a cleaning and disinfection device for reprocessing a surgical instrument.

23. The method according to claim 13, wherein the controller is configured to one or more of:
open a door of the reprocessing device upon the at least one recognition of the at least one non-contact gesture of the reprocessing device operator detected by the sensor; and
close the door of the reprocessing device upon the at least one recognition of the at least one non-contact gesture of the reprocessing device operator detected by the sensor.

24. The method according to claim 13, wherein the controller is configured to one of:
perform a reprocessing procedure for reprocessing the surgical instrument upon the at least one recognition of the at least one non-contact gesture of the reprocessing device operator; or
stop the reprocessing procedure for reprocessing the surgical instrument upon the at least one recognition of the at least one noncontact gesture of the reprocessing device operator.
